(19) United States
(12) Patent Application Publication
(10) Pub. No.: US 2015/0147243 A1
(43) Pub. Date: May 28, 2015

(54) MIXED MULTIFUNCTIONAL METAL AFFINITY SURFACES FOR REDUCING AGGREGATE CONTENT IN PROTEIN PREPARATIONS

(71) Applicant: Agency for Science, Technology and Research, Connexis (SG)

(72) Inventor: Peter Gagnon, Centros (SG)

(21) Appl. No.: 14/555,165

(22) Filed: Nov. 26, 2014

Related U.S. Application Data
(63) Continuation of application No. PCT/SG2013/000048, filed on Feb. 6, 2013.
(60) Provisional application No. 61/653,716, filed on May 31, 2012.

(51) Int. Cl. C07K 16/32; B01D 15/38; B01D 15/34; C07K 16/00; B01D 15/36

(52) U.S. Cl. CPC C07K 16/32; C07K 16/00; B01D 15/362; B01D 15/34; B01D 15/363; B01D 15/3809
USPC 422/261; 252/184; 428/364

(57) ABSTRACT

Compositions for reducing the aggregate content of a protein preparation include a first substrate having a first surface-bound ligand possessing a metal affinity functionality and a second surface-bound ligand optionally provided on a second substrate and having an aggregate charge opposite to that of the metal affinity functionality of the first substrate, wherein the first surface-bound ligand and the second surface-bound ligand are positioned such that the protein preparation may contact both the first surface-bound ligand and the second surface-bound ligand simultaneously.

### CROSS-REFERENCE TO RELATED APPLICATIONS

This application claims the priority of PCT/SG2013/000048 filed Feb. 6, 2013, which claims the priority of U.S. Provisional Application No. 61/653,716, filed May 31, 2012 the disclosures of which are incorporated herein by reference in their entireties.

### FIELD

This invention relates to materials for purification of proteins, especially antibodies. It particularly relates to materials for reducing the content of aggregates, and especially aggregates that include host cell chromatin remnants such as nucleosomes, histones, and DNA.

### BACKGROUND

It has been indicated that unnatural hetero-aggregates form spontaneously between host cell-derived contaminants and recombinant proteins produced by in vitro cell culture methods (Shukla et al., Biotechnol. Progr. (2008) 24:1115-1121; Luhrs et al., J. Chromatogr. B (2009) 877:1543-1552; Mechetner et al., J. Chromatogr. B (2011) 879:2583-2594; Gagnon et al., J. Chromatogr. A, (2011) 1218:2405-2412; Gagnon, Bioprocessing J. (2010) 9(4):14-24). These hetero-aggregates may be considered unnatural in two respects: 1) constituent contaminants are often of non-human origin, secreted by living non-human host cells or released into the culture media when non-human host cells lyse upon death. In living humans, such non-human contaminants do not exist; and 2) constituent contaminants accumulate to high concentrations in comparison to human in vivo systems where dead cell constituents are quickly eliminated. Accordingly, recombinant products are exposed to high levels of strongly interactive contaminants at concentrations that typically do not occur in living systems. Meanwhile, high expression levels of recombinant proteins make them suitable substrates for non-specific associations with these non-human contaminants, favoring the formation of undesirable hetero-aggregates of diverse composition.

The contaminating protein content of hetero-aggregates has been addressed to some extent via direct targeting of the contaminating protein (Shukla et al. and Gagnon et al. supra), as well as indirectly via targeting of the corresponding DNA component responsible for the contaminating protein (Luhrs et al. and Gagnon supra). A reduction of antibody aggregate level has been indicated when some complexes are dissociated (Shukla et al., Mechetner et al., and Gagnon supra). The ability of anion exchangers to reduce levels of antibody-contaminant complexes has been disclosed (Luhrs et al. and Gagnon et al. supra), but an anion exchange treatment that was able to fully eliminate hetero-aggregates has not been indicated. Size exclusion, cation exchange, and hydrophobic interaction chromatography have also been employed in attempts to reduce hetero-aggregates, but these techniques were generally inferior to anion exchange (Gagnon et al. supra).

The specific source of contaminants that form stable associations with antibodies is not always known (see, for example, Shukla et al. supra). Some efforts have focused on DNA contaminants with little attention to the specific source of other possible contaminants (Gagnon et al. and Gagnon supra). Some efforts indicating an association of host contaminants with aggregates in antibody preparation have focused specifically on contaminants comprising chromatin catabolites (Luhrs et al. and Mechetner et al. supra). In these examples, aggregation may be mediated directly through the immunospecificity of the antibody for chromatin catabolites such as histones and DNA. It has been indicated that chromatin catabolites are also capable of forming stable complexes with antibodies via non-specific interactions. Thus, monoclonal antibodies with known immunospecificities for antigens not including chromatin catabolites, can form highly stable aggregates of diverse descriptions with nucleosomes, histones, and DNA derived from the nuclei of dead host cells. It has been particularly indicated that chromatin catabolites are highly represented in high molecular weight (HMW) aggregates. HMW aggregates are of particular concern because of their suspected involvement in promoting the formation of therapy-neutralizing antibodies. HMW aggregates are generally defined as aggregates of a size greater than small multiples of the antibody of interest. For example, 2-antibody associations are not considered HMW aggregates, nor are most 4-antibody aggregates. However, aggregates of much greater size, such as corresponding to about 8 to about 10 or more antibodies may be generally classified as HMW aggregates.

Treating antibody preparations with agents that might be expected to dissociate hetero-aggregates has generally proven ineffective. For example, employing high concentrations of urea, salts, or combinations of the two does not substantially dissociate IgM-contaminant hetero-aggregates (Gagnon et al. supra). Protein A affinity chromatography with pre-elution washes of urea, alcohol, and surfactants has been indicated to reduce hetero-aggregate levels more effectively than without washes (Shukla et al. supra), as did pre-elution washes combining urea, salt, and EDTA with protein G affinity chromatography (Mechetner et al. supra). Anion exchange chromatography with a pre-elution wash of urea has been indicated to reduce hetero-aggregates more effectively than in the absence of a urea wash (Gagnon et al. supra). Cation exchange chromatography has also been indicated to reduce hetero-aggregates more effectively with a pre-elution EDTA wash than without the wash (Gagnon et al. supra). Finally, hydroxyapatite with pre-elution washes of urea and/or salt have also reduced hetero-aggregates more effectively than without such washes (Gagnon supra). Despite these observations, in general, the use of dissociating agents in pre-elution washes of antibodies bound to chromatography columns has been only moderately successful.

### SUMMARY

The invention provides in certain embodiments, compositions of matter and apparatus for the purification of proteins, including the reduction of aggregate concentration in antibody preparations. In certain embodiments, the compositions particularly reduce the content of aggregates that include chromatin remnants, such as nucleosomes, and/or histones, and/or DNA. In certain embodiments, a composition of matter for reducing the aggregate content of a protein preparation is provided where the composition includes a first surface-bound ligand possessing metal affinity functionality and a second surface-bound ligand having an aggregate charge opposite to that of the metal affinity functionality of the first surface and where the first surface-bound ligand and the second surface-bound ligand are positioned such that a protein preparation may contact both the first surface-bound ligand and the second surface-bound ligand simultaneously.

### BRIEF DESCRIPTION OF THE DRAWINGS

FIG. 1 shows a time course plot of the size exchange chromatography (SEC) profiles sampled at indicated intervals in the purification of IgM-529 which was subjected to allantoin-ethacridine batch particle treatment. Aggr: aggregate. HCP: host cell protein. Solid line: 280 nm. Broken line: 254 nm. All time scales same as center panel.

FIG. 2A shows an SEC profile of IgM-84 filtered cell culture supernatant (CCS). Aggr: aggregates. HMW: high molecular weight aggregates. HCP: host cell protein. LMW: low molecular weight cell culture media components. Solid line: 280 nm. Broken line: 254 nm.

FIG. 2B shows an SEC profile of the IgM-84 filtered CCS of FIG. 2A after allantoin-ethacridine and column flow through treatment. Aggr: aggregate. HCP: host cell protein. Solid line: 280 nm. Broken line: 254 nm.

FIG. 3 shows an SEC profile of monoclonal IgG HER2 CCS before (left panel) and after (right panel) treatment with allantoin-ethacridine and column flow through treatment. Aggr: aggregate. HCP: host cell protein. LMW: low molecular weight cell culture media components. LC: light chain. Solid line: 280 nm. Broken line: 254 nm.

### DETAILED DESCRIPTION

It has been surprisingly discovered that in certain embodiments of the invention a composition of matter comprising a combination an electronegative surface-bound metal affinity ligand (first surface-bound ligand) with an electropositive surface-bound ligand (second surface-bound ligand), or alternatively comprising a combination an electropositive surface-bound metal affinty ligand (first surface-bound ligand) and an electronegative surface-bound ligand (second-surface-bound ligand), has the ability to substantially reduce the content of high molecular weight (HMW) aggregates and aggregates of smaller size in protein preparations. In certain embodiments, the first surface-bound ligand and the second surface-bound ligand may reside on the same surface. In certain embodiments the first surface-bound ligand and the second surface-bound ligand may reside on separate surfaces. In certain embodiments the first surface-bound ligand and the second surface-bound ligand may reside or either the same surface and/or separate surfaces. Certain embodiments of the invention have the additional ability to remove agents such as multivalent ions and antiviral compounds that may have been added to the protein preparation.

In certain embodiments the invention provides devices comprising a mixture of two or more surface functionalities for reducing aggregate levels in protein preparations, where at least one of the surfaces has a metal affinity functionality with the ability to form stable coordination bonds with metal ions, and the other surface has a net charge opposite to the metal affinity functionality. In a preferred embodiment, the invention may provide compositions having one surface with an electronegative metal affinity functionality, while another surface embodies an electropositive metal affinity functionality. Either surface may embody or be combined with additional chemical functionalities, potentially including but not limited to hydrophobic, pi-pi bonding, hydrogen bonding, and metal affinity. The solid surfaces may be particulate, fibrous, porous-membranaceous, or monolithic in structure, including combinations of multiple structural types. The device may be configured in such a way that contact of an applied sample with the electronegative and electropositive surfaces is simultaneous.

The invention provides in certain embodiments, compositions of matter and apparatus for the purification of proteins, including the reduction of aggregate concentration in antibody preparations. In certain embodiments, a composition of matter for reducing the aggregate content of a protein preparation is provided where the composition includes a first surface-bound ligand possessing metal affinity functionality and a second surface-bound ligand having an aggregate charge opposite to that of the metal affinity functionality of the first surface and where the first surface-bound ligand and the second surface-bound ligand are positioned such that a protein in a protein preparation may contact both the first surface-bound ligand and the second surface-bound ligand simultaneously.

In certain embodiments, the first surface-bound ligand and the second surface-bound ligand are each covalently bound to a substrate. The first surface-bound ligand and the second surface-bound ligand may be each covalently bound to the same substrate. In other embodiments, the first surface-bound ligand and the second surface-bound ligand are each covalently bound to different substrates. The substrate may be a particle and in certain embodiments the particle may be porous or non-porous. In certain embodiments, the substrate is a porous particle having pores large enough to permit entry of a protein in a protein preparation. In other embodiments, the pores are too small to permit entry of a protein in a protein preparation. In certain embodiments the substrate is porous particle having an average pore size between about 10 nm and about 100 nm, less than about 10 nm, or above about 100 nm. In certain embodiments, the substrate is a membrane, a monolith, or a solid or porous walled fiber. In certain embodiments the substrate to which the first chemical moiety is bound is of a different kind than the substrate to which the second moiety is bound. For example one substrate may be a particle while the other substrate may be a monolith, membrane or fiber.

In certain embodiments, the first surface-bound ligand and the second surface-bound ligand are different. In certain embodiments, the first surface-bound ligand is a multidentate metal chelating moiety. In certain embodiments, the first surface-bound ligand has an aggregate charge which is electronegative. In others, the first surface-bound ligand has an aggregate charge which is electropositive. In certain embodiments, the second surface-bound ligand possesses metal affinity functionality. In certain such embodiments, the second surface-bound ligand is a multidentate metal chelating moiety.

In certain embodiments, the first surface-bound ligand is electropositive and that surface has additional chemical moieties bound to it, provided that the aggregate charge of that surface is electropositive. In others, the first surface-bound ligand is electronegative and that surface has additional chemical moieties bound to it, provided that the aggregate charge of that surface is electronegative. In certain embodiments, at least one of the substrates has one or more chemical moieties in addition to the first surface-bound ligand or the second surface-bound ligand wherein such additional chemical moieties enhance the capacity of the composition to participate in hydrogen bonding, hydrophobic interactions, or pi-pi binding with a protein of the protein preparation.

In certain embodiments, the first surface-bound ligand is electronegative and is iminodiacetic acid (2-(carboxymethylamino)acetic acid), ethylene glycol(aminoethylether) diacetic acid, nitriloacetic acid (2,2′,2″-Nitrilotriacetic acid), aspartic acid, or glutamic acid. In certain embodiments, the first surface-bound ligand is electropositive and is tris(2-aminoethyl)amine or desferoxamine. In certain of such embodiments, the second surface-bound ligand is tris(2-aminoethyl)amine. In certain embodiments, the first surface-bound ligand is iminodiacetic acid (2-(carboxymethylamino)acetic acid) and the second surface-bound ligand is tris(2-aminoethyl)amine.

In certain embodiments, the surface-bound chemical moieties described in the foregoing embodiments may alternatively be directly incorporated in the structure of the polymer or polymers during synthesis of the physical surface.

In certain embodiments, the invention provides an apparatus configured for chromatography including a composition of the invention. In certain embodiments, the apparatus is a chromatographic column packed with the substrate or substrates to which the first surface-bound ligand and the second surface-bound ligand are bound. In certain embodiments, the apparatus contains one or more porous membranes and at least one of such membranes is the substrate to which the first surface-bound ligand and the second surface-bound ligand are bound. In certain embodiments, the apparatus contains a porous reticular arrangement of fibers, where such fibers are hollow porous-walled fibers or non-porous fibers and such fibers are the substrates to which the first surface-bound ligand and the second surface-bound ligand are bound.

In certain embodiments, the apparatus contains porous or non-porous particles sandwiched between porous membranes or monoliths. In certain such embodiments, the particles are the substrate to which either the first surface-bound ligand or the second surface-bound ligand is bound and the membrane or monolith is the surface to which the other of the first surface-bound ligand and the second surface-bound ligand is bound. In certain embodiments, the apparatus contains porous or non-porous particles sandwiched between woven or amorphous fibrous filters. In certain such embodiments, the particles are the substrate to which either the first surface-bound ligand or the second surface-bound ligand is bound and the fibrous filters are the surface to which the other of the first surface-bound ligand and the second surface-bound ligand is bound. In certain embodiments, the apparatus contains porous or non-porous particles sandwiched between crystalline frits. In certain such embodiments, the particles are the substrate to which either the first surface-bound ligand or the second surface-bound ligand is bound and the crystalline frits are the surface to which the other of the first surface-bound ligand and the second surface-bound ligand is bound. In certain embodiments, the apparatus contains porous or non-porous particles embedded in a reticular polymer network. In certain such embodiments, particles are the substrate to which either the first surface-bound ligand or the second surface-bound ligand is bound and the reticular polymer network is the surface to which the other of the first surface-bound ligand and the second surface-bound ligand is bound. In certain embodiments, the apparatus provides where both the substrate to which the first surface-bound ligand is bound and the substrate to which the second surface-bound ligand is bound are both particles and the particles are confined between membranes, monoliths, a reticular polymer network, woven or amorphous fiber filters, crystalline frits, or a combination thereof.

In certain embodiments, the chemical surface of one or more components of the apparatus may be relatively inert or of such a relatively low surface area as to make no significant contribution to the chemical functionality of the apparatus. In certain such embodiments, the chemical surface which is relatively inert or of relatively low surface area is configured so as to create structural integrity, or direct flow of liquids therethrough, or physically block, entrap, or entrain insoluble materials in a protein preparation to prevent them from interfering with the effective use of the device.

In some embodiments, a ratio of a first surface and a second surface may be in a range of from about 1:99 to about 99:1. Those skilled in the art will appreciate that almost any ratio may be appropriate for a particular application. As a matter of practicality, in some embodiments, optimizing a ratio of first surface to second surface may begin by employing an equitable 1:1 ratio of the first and second components and optimizing the ratio systematically by altering the ratios from this starting point.

Terms are defined so that the invention may be understood more readily. Additional definitions are set forth throughout the detailed description.

“Aggregate(s)” refers to an association of two or more molecules that is stable at physiological conditions and may remain stable over a wide range of pH and conductivity conditions. Aggregates frequently comprise at least one biomolecule such as a protein, nucleic acid, or lipid and another molecule or metal ion. The association may occur through any type or any combination of chemical interactions. Aggregates of antibodies can be classified into two categories: “Homoaggregates” refers to a stable association of two or more antibody molecules; “Hetero-aggregates” refers to a stable association of one or more antibody molecules with one or more non-antibody molecules. The non-antibody component may consist of one more entities from the group consisting of a nucleotide, an endotoxin, a metal ion, a protein, a lipid, or a cell culture media component.

“Antibody” refers to an immunoglobulin, composite, or fragmentary form thereof. The term may include but is not limited to polyclonal or monoclonal antibodies of the classes IgA, IgD, IgE, IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” may also include composite forms including but not limited to fusion proteins containing an immunoglobulin moiety. “Antibody” may also include antibody fragments such as Fab, F(ab′)2, Fv, scFv, Fd, dAb, Fc and other compositions, whether or not they retain antigen-binding function.

“Endotoxin” refers to a toxic heat-stable lipopolysaccharide substance present in the outer membrane of gram-negative bacteria that is released from the cell upon lysis. Endotoxins can be generally acidic due to their high content of phosphate and carboxyl residues, and can be highly hydrophobic due to the fatty acid content of the lipid-A region. Endotoxins can offer extensive opportunity for hydrogen bonding.

“Substrate” or “Solid material” refers to an insoluble organic or inorganic solid that may be particulate, crystalline, polymeric, fibrous, porous-hollow fibrous, monolithic, or membranaceous in nature. It may consist of non-porous or porous particles, a porous membrane, a porous filter, or a porous monolith. If particulate, the particles may be roughly spherical or not, and may be of sizes ranging from less than 100 nm to more than 100 microns. The average pore size of porous particles may range less than 10 nm (microporous) to more than 100 nm (macroporous). The average pore size in membranes may range from less than 100 nm to more than 1 micron. The average channel size in membranes or monoliths may range from less than 1 micron to more than 10 microns. The solid material may further consist of compound constructions, for example in which particles are embedded in a reticular matrix, sandwiched between membranes, or both.

“Metal affinity functionality” refers to the capacity of a chemical moiety, which may be immobilized on a surface, to bind metal ions preferably in a 1:1 fashion. Such moieties may have the capacity to form coordination bonds with a metal ion and certain such moieties may be bidentate or multidentate in character. Nonlimiting examples of electronegative moieties with this capability include iminodiacetic acid (2-(carboxymethylamino)acetic acid), diethylamine triamine pentacetic acid, and nitriloacetic acid (2,2′,2″-nitrilotriacetic acid). Examples of electropositive compounds with this capability include but are not limited to Tris(2-aminoethyl)amine, diethylenetriamine, triethylenetetramine, tetraethylenepentamine, polypropylenimine tetraamine, and desferrioxamine.

“Electropositive surface” refers to a surface of a substrate or solid material which is dominated by positive charge. Electropositivity of a surface may be conferred by chemical groups including but not limited to weak anion exchange groups, like amino, ethylene diamino, diethylaminoethyl, polyallylamine, polyethyleneimine, strong anion exchange groups, such as quaternary amino groups, combined weak-strong exchangers, such as polylysine, polyarginine, or Tris(2-aminoethyl)amine, diethylenetriamine, triethylenetetramine, tetraethylenepentamine, polypropylenimine tetraamine, PAMAM dendrimer (ethylenediamine core), or any combinations of the foregoing. Secondary functionalities that create a mixed chemical character on a positively charged surface may consist of negatively or positively charged groups, hydrophobic groups, pi-pi bonding groups, hydrogen-bonding groups, or metal-chelation groups. The secondary functionalities may exist on electropositive surfaces as an inadvertent byproduct of the manufacturing materials or process by which the particles are synthesized, or they may be present by deliberate design. The concentration of secondary functionalities may range from less than 1 milliequivalent per mL of particles, to more than 100 milliequivalents per mL.

“Electronegative surface” refers to a surface of a substrate or solid material which is dominated by negative charge. Electronegativity of a surface may be conferred by chemical groups including but not limited to so called weak cation exchangers, such as carboxyl, aminocarboxyl (iminodiacetic or nitriloacetic), or phosphoryl, or strong exchangers such as sulfo groups (e.g., sulfo, sulfomethyl, sulfoethyl, sulfopropyl). Secondary functionalities that create a mixed chemical character on a negatively charged surface may consist of negatively or positively charged groups, hydrophobic groups, pi-pi bonding groups, hydrogen-bonding groups, or metal-chelation groups. The secondary functionalities may exist on electronegative surfaces as an inadvertent byproduct of the manufacturing process by which the particles are synthesized, or they may be present by deliberate design. The concentration of secondary functionalities may range from less than 1 milliequivalent per mL of particles, to more than 100 milliequivalents per mL.

“Polynucleotide” refers to a biopolymer composed of multiple nucleotide monomers covalently bonded in a chain. DNA (deoxyribonucleic acid) and RNA (ribonucleic acid) are examples of polynucleotides. Polynucleotides can have a high propensity for formation of hydrogen bonds.

“Protein” refers to any of a group of complex organic macromolecules that contain carbon, hydrogen, oxygen, nitrogen, and usually sulfur and are composed principally of one or more chains of amino acids linked by peptide bounds. The protein may be of natural or recombinant origin. Proteins may be modified with non-amino acid moieties such as through glycosylation, pegylation, or conjugation with other chemical moieties. Examples of proteins include but are not limited to antibodies, clotting factors, enzymes, and peptide hormones.

“Protein preparation” refers to any aqueous or mostly aqueous solution containing a protein of interest, such as a cell-containing cell culture harvest, a (substantially) cell-free cell culture supernatant, or a solution containing the protein of interest from a stage of purification.

“Virus” or “virion” refers to an ultramicroscopic (roughly 20 to 300 nm in diameter), metabolically inert, infectious agent that replicates only within the cells of living hosts, mainly bacteria, plants, and animals: composed of an RNA or DNA core, a protein coat, and, in more complex types, a surrounding envelope.

In certain embodiments, the solid materials used to practice the invention may include insoluble particles of natural or synthetic origin, such as but not limited to porous microparticles commonly employed for practicing chromatography. Such particles may embody large pores that permit the diffusive entry of proteins, such as but not limited to antibodies; or they may embody small pores that allow the diffusive entry of small chemical species such as salts, sugars, and hetero-aggregate-dissociating agents, but are too small to permit the entry of proteins such as antibodies. The solid materials may alternatively include non-porous particles, membranes or monoliths, fibers including porous-walled hollow fibers, porous membranes, and/or compound constructions employing combinations of the above elements.

Electropositive groups may include so-called strong anion exchange groups and/or so-called weak anion exchange groups. The term strong anion exchanger includes functional groups such as quaternary amines, which embody pKas above pH 12. The term weak anion exchanger is understood to refer to functional groups such as diethylaminoethyl, and ethylenediamine, which embody pKas lower than 12. Electropositive groups of weak or mixed strong-weak anion exchange may be preferred for their ability to participate in coordination interactions with dissolved metal ions, producing the desirable result of removing contaminating metal ions from the applied biological sample. A non-limiting example of such a mixed weak-strong anion exchange group with metal coordination ability is Tris(2-aminoethyl)amine (TREN).

Electronegative groups may include so-called strong cation exchange groups or so-called weak cation exchange groups. The term strong cation exchanger is understood to include sulfate or sulfo containing moieties with pKas below 3. The term weak cation exchanger is understood to include moieties containing carboxy and/or phospho groups with pKas above 3. Dominantly electronegative groups of dipolar character (at neutral pH), such as combinations of two carboxyl groups with an amino group may be preferred for their strong ability to participate in coordination interactions with dissolved metal ions, producing the desirable result of removing contaminating metal ions from the applied biological sample. Non-limiting examples of such groups include iminodiacetic (IDA), and nitriloacetic acid (NTA) groups.

The surfaces of the electropositive or electronegative materials may also incorporate hydrophobic groups of an aliphatic and/or or aromatic character, where the latter may be preferred because of their ability to participate in so-called pi-pi binding. Mixed chemical character may reside in a single complex chemical group, in separate chemical groups of distinct character on a single type of surface, on distinct surfaces, or any combination of the foregoing. One or more electronegative metal affinity and/or one or more electropositive metal affinity groups may be employed simultaneously, and they may differ with respect to the form in which they are embodied. Chemical functionalities of differing individual character may be employed in various ratios customized to the needs of a particular sample composition. For example, combinations intended for treatment of clarified cell culture supernatant may include an excess of electropositive surfaces, while combinations intended for treatment of supernatant already treated with electropositive hetero-aggregate-dissociating agents such as ethacridine or polyethyleneimine may include an excess of electronegative surfaces.

In certain embodiments, the invention may provide particles with an electronegative metal affinity functionality, combined with electropositive particles which may be mixed together and which may further be mixed with and/or enclosed by neutral materials, embedded in neutral materials, or enclosed by or embedded in materials that are themselves electronegative and/or electropositive.

In certain embodiments, the invention may provide particles with an electropositive metal affinity functionality, combined with electronegative particles which may be mixed together and which may further be mixed with and/or enclosed by neutral materials, embedded in neutral materials, or enclosed by or embedded in materials that are themselves electronegative and/or electropositive.

In certain embodiments, the invention may provide particles with an electronegative metal affinity functionality, combined with particles with an electropositive metal affinity functionality which may be mixed together and which may further be mixed with and/or enclosed by neutral materials, embedded in neutral materials, or enclosed by or embedded in materials that are themselves electronegative and/or electropositive.

In certain embodiments, the invention may provide electronegative and/or electropositive surfaces which may embody additional chemical functionalities, including but not limited to the ability to participate in hydrophobic interactions, pi-pi bonding, hydrogen bonding, and metal affinity. In certain embodiments, the invention may provide one or more types of electronegative particles and one or more types of electropositive particles mixed together and enclosed by neutral materials.

In certain embodiments, the particles may have differing sizes and/or differing porosities. In certain embodiments, the invention may provide particles which may be mixed with and or enclosed between electronegative and/or electropositive materials of other physical form, including but not limited to membranes, fibers, and monoliths.

In certain embodiments, differing proportions of electronegative and electropositive functionalities on one or more substrates may be selected to accommodate the needs of protein preparations of differing composition.

It will be apparent to the person of ordinary skill in the art that, in addition to being useful for reducing the content of homoaggregates and hetero-aggregates in a protein preparation, that in certain embodiments, the invention may be useful for substantially reducing the content of host cell protein, polynucleotides, endotoxin, and virus from a protein preparation. In the case that the primary functionalities are accompanied by additional functionalities, such as hydrophobic and hydrogen bonding for example, the invention may also reduce content of cell culture media components and additives that limit the ability of downstream purification methods to reproducibly achieve their goals. It will also be apparent to the person of ordinary skill, that although certain embodiments of the invention may have substantial value when applied to relatively crude feed streams, it may nevertheless offer, in certain embodiments, important value when to applied samples that are substantially purified.

In certain embodiments, the invention may provide compositions or apparatus such that the solid materials may be cleaned and recycled after use. In other embodiments, they may be configured for single use.

Additional objects and advantages of the invention will be set forth in part in the description which follows, and in part will be obvious from the description, or may be learned by practice of the invention. The objects and advantages of the invention will be realized and attained by means of the elements and combinations specified in the claims.

It is to be understood that both the foregoing general description and the following detailed description are exemplary and explanatory only and are not restrictive of the invention, as claimed.

### EXAMPLES

### Example 1

A prototype particle mixture was prepared from 0.5 mL of negatively charged chelating porous particles (Chelex-100) and 0.5 mL electropositive porous particles (Macroprep High-Q), mixed and sandwiched between cellulose filters in a glass assembly. A purified sample of IgG monoclonal antibody (Her2) was passed over the particles without loss at near-physiological conditions: pH 7.0, 10 mS/cm. Experiments with unpurified antibody were conducted at 25 mS/cm, 20 mS/cm, and 30 mS/cm. Hetero-aggregates and HMW aggregates were removed in all cases. Experiments with unpurified antibody containing 0.02% ethacridine were conducted at the same conductivities. Hetero-aggregates and HMW aggregates were removed in all cases, and the flow-through was free of ethacridine as documented by the lack of UV absorbance at 365 nm.

### Example 2

In a set of experiments parallel with Example 1, a purified sample of IgM monoclonal antibody (clone 529) was passed over the particles at elevated salt conditions: pH 7.0, 20 mS/cm. Unpurified antibody, and unpurified antibody containing 0.02% ethacridine were applied at 20, 30, and 40 mS/cm. Hetero-aggregates and HMW aggregates were removed in all cases, and the flow-through was free of ethacridine as documented by the lack of UV absorbance at 365 nm. These experiments were repeated with 0.2% ethacridine added to the sample. Hetero-aggregates and HMW aggregates were removed in all cases, and the flow-through was free of ethacridine.

### Example 3

Capacity study of the removal of DNA and aggregates from a monoclonal IgM culture supernatant when passed over a packed bed consisting of an equivolume mixture of Chelex-100 and MacroPrep High Q. A 0.5 mL each of Chelex-100 and MacroPrep High Q were sandwiched between PVDF membranes. IgM-529 cell culture supernatant was loaded at 1 mL/min and samples of the flow-through taken at 25, 50, and 100 mL, then analyzed by analytical SEC. Even at maximal loading, 254-dominant hetero-aggregates were essentially eliminated from the flow-through. IgM recovery at the various loads (low-to-high) was 58, 73 and 85% respectively. The experiment was repeated with NaCl added to produce a conductivity of 25 mS/cm. Heteroaggregate reduction was unaffected but IgM recovery increased to about 85, 90, and 95% respectively.

### Example 4

The experiment of Example 3 was repeated but at a conductivity of 20 mS/cm with a combination of microporous and macroporous electropositive and electronegative media, plus microporous lipophilic particles in equal volumes: QAE Sephadex A-25, SP Sephadex C-25, Nuvia Q, Nuvia S and Sephadex LH-20. Allantoin and ethacridine were added to 5% serum-supplemented monoclonal IgM supernatant, to final concentrations of 1% (super-saturation) and 0.02% respectively. The supernatant was clarified by centrifugation and then flowed through the bed (20 mL supernatant per mL packed bed). The IgM was then captured and fractionated by cation exchange chromatography. IgM recovery was 80% over the two-step process and the purity was more than 90% by analytical SEC, with no apparent aggregates.

### Example 5

An equal mixture of microporous styrenedivinylbenzene particles bearing negatively charged metal-chelating iminodiacetic acid groups and macroporous agarose particles bearing the positively charged chelating ligand tris(2-aminoethyl)amine was sandwiched between polyethylene fits and equilibrated with 50 mM Hepes, 100 mM NaCl, pH 7.0. Filtered mammalian cell culture supernatant containing IgG (Clone HER2) was passed through the assembly and analyzed size exclusion chromatography. The untreated sample contained about 10% aggregates. The treated sample contained less than 0.2% aggregates. Testing with AccuBlue revealed that treatment also removed 98% of the DNA. Antibody recovery was 99%.

### Example 6

The particle mixture of Example 5 was equilibrated to 50 mM Hepes, 100 mM NaCl, pH 7.0 and added directly to the filtered mammalian cell culture supernatant containing IgG (Clone HER2), incubated stirring for 1 hour, then removed by membrane filtration. Analysis revealed roughly half the aggregate reduction and DNA removal of example 5. Incubation stirring for 16 hours at 4 exhibited about 80% the efficiency of Example 5.

### Example 7

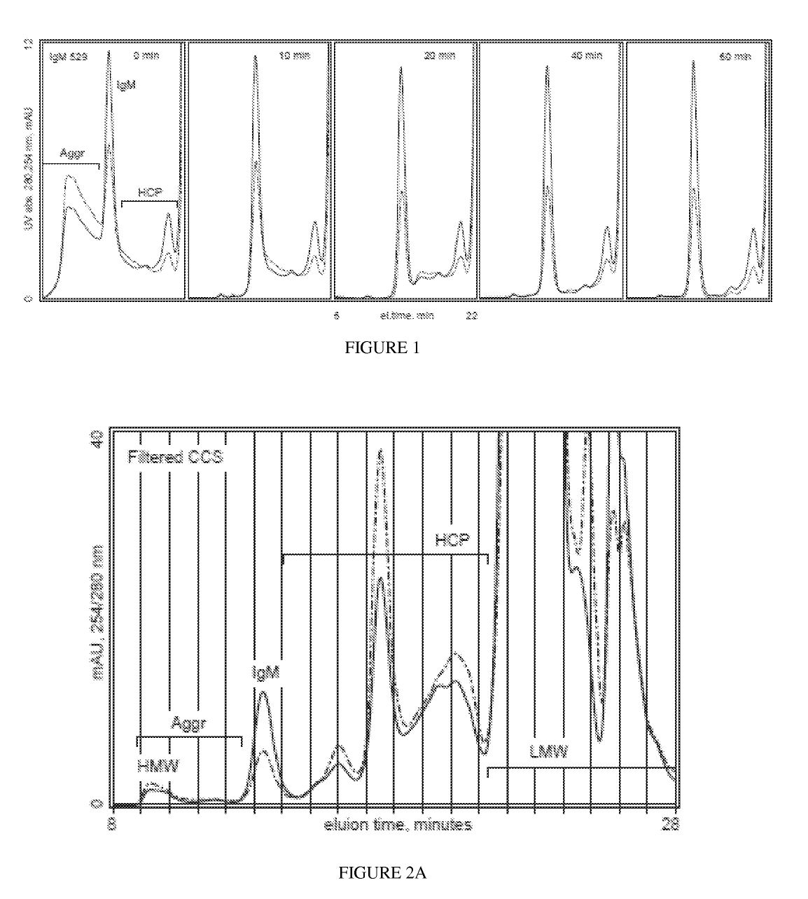

An equal mixture of negatively charged metal-chelating styrene divinylbenzene particles, positively charged polymethacrylate porous particles, and negatively charged polymethacrylate particles was mixed with a sample of IgM-529 that had been previously treated with NaCl to a final conductivity of 20 mS/cm, 1% allantoin, and 0.025% ethacridine. The volumetric ratio of particles to sample was 1:20. Samples were taken at 10 minutes, 20 minutes, 40 minutes, and 60 minutes, and the particles were removed by microfiltration. FIG. 1 shows a dramatic reduction of high molecular weight aggregates at all time points, but with progressively greater reduction of all aggregates over time, accompanied by a substantial apparent reduction of host cell protein contaminants as well. Subsequent analysis showed that the reduction of both aggregates and host proteins reflected the combined reduction of chromatin remnants from the sample. Ethacridine was also removed from the samples at all time points.

### Example 8

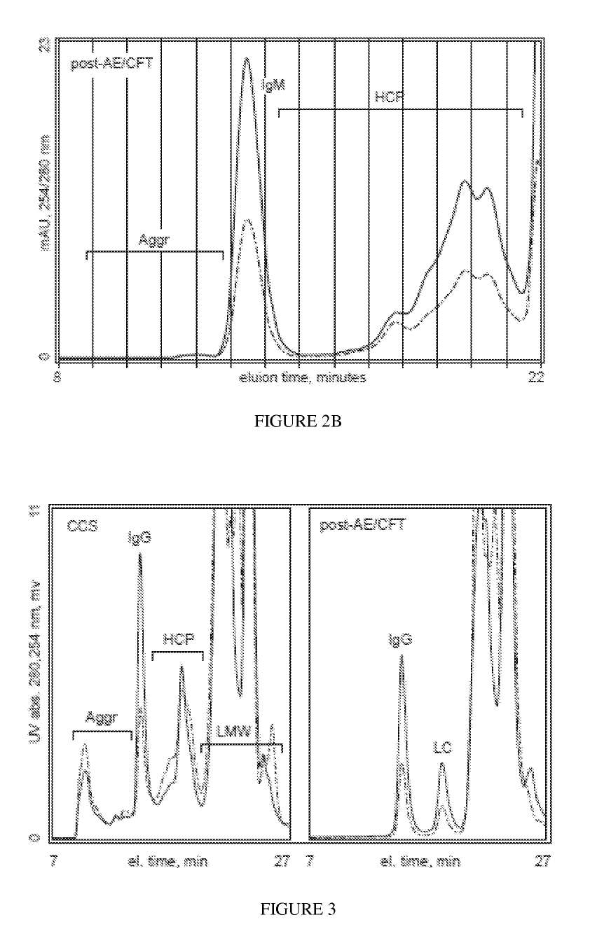

FIGS. 2A and 2B illustrate size exclusion chromatography profiles before and after treatment of IgM-84 treated with NaCl, allantoin, and ethacridine as in Example 7, and then treated with the same media mixture as in Example 7, but by passing the sample though a device in which the mixed media were sandwiched between woven polymer retainers of adequately narrow mesh to retain the particles. HMW aggregate was completely eliminated, along with the majority of smaller aggregates. Table 1 below shows that DNA and histones were initially distributed across all aggregate fractions, with the IgG fraction, and across all protein-containing fractions.

TABLE 1
IgM and contaminant content of SEC fractions.

| ElT, min | [IgM] | DNA size | [DNA] | [His] | 254:280 |
|---|---|---|---|---|---|
| 9 | 0.31 | bld | 10 | 0.13 | 1.31 |
| 10 | 0.09 | bld | 10 | 0.11 | 1.30 |
| 11 | 0.42 | bld | 10 | 0.13 | 1.03 |
| 12 | 0.68 | (150-1000) | 12 | 0.13 | 0.98 |
| 13 | 20.29 | bld | 44 | 0.21 | 0.49 |
| 14 | 21.08 | (660) | 236 | 0.76 | 0.89 |
| 15 | 2.33 | 445/(660) | 459 | 1.87 | 0.97 |
| 16 | 0.30 | 316/445 | 435 | 0.82 | 1.59 |
| 17 | 0.51 | 316 | 796 | 1.09 | 1.45 |
| 18 | 0.38 | 155/316 | 314 | 0.96 | 1.60 |
| 19 | 0.31 | 90/155 | 339 | 0.33 | 1.40 |
| 20 | 0.09 | 90 | 684 | 0.86 | 1.56 |
| 21 | bld | 58 | 123 | 1.33 | 0.90 |
| 22 | bld | bld | 23 | 0.54 | 1.21 |
| 23 | bld | bld | 13 | bld | 3.67 |
| 24 | bld | bld | 3 | bld | 15.86 |

ElT: elution time, minutes.
[IgM]: concentration in micrograms/mL.
DNA size in base pairs (bp).
Values in parentheses from DNA detected in ion exchange experiments. [DNA] concentration in ng/mL.
[His]: total histone concentration, micrograms/mL.
bld: below limit of detection.

Table 1 also illustrates the size distribution of DNA, which led to the unexpected discovery that some aggregate populations included nucleosomal arrays containing various numbers of nucleosomes, in addition to DNA and histones. Recovery of IgM from this treatment was 98%.

### Example 9

FIG. 3 illustrates size exclusion chromatography profiles before and after the same procedure of Example 16 of an anti-HER2 monoclonal IgG antibody. The results are the same in kind, but improved in degree over Example 8, likely, at least in part, because the concentration of the antibody is about 10 times higher.

### Example 10

IgM-84 cell culture supernatant with NaCl added to a conductivity of 20 mS/cm was passed through a porous monolithic polymethacrylate disk (CIM IDA, BIA Separations) bearing the surface-bound negatively-charged chelating agent iminodiacetic acid, then immediately through a monolith bearing diethylaminoethyl positively charged groups (CIM DEAE, BIA), a volumetric ratio of 50 to 1, supernatant:monoliths. Aggregates and DNA were removed as effectively as in Example 7. IgM recovery was 98%. Histone and general host protein removal were about half the level achieved in Example 7, both at about 35% versus the approximate 70% from Example 7. It will be apparent to the person of skill that both chemical groups could alternatively reside mixed on the surface of a single monolith.

### Example 11

The procedure of Example 10 was repeated except replacing the positively charged monolith with a microfiltration membrane bearing positively charged quaternary amino groups (Sartobind Q nano). DNA and aggregate removal, and IgM recovery were unchanged. It will be apparent to the person of skill that both chemical groups could reside on the surface of a single membrane.

### Example 12

The procedure of Example 11 were repeated except replacing the positively charged membrane with a positively charged microporous hollow fiber (Qyu-speed D, Asahi-Kasei Medical Company) with grafted ligands bearing derivatized amino ligands in an extended configuration.

### Example 13

The procedure of Example 12 was reproduced except using porous agarose particles bearing the positively charged chelating agent tris(2-aminoethyl)amine (BioWorks TREN hi-sub) sandwiched between woven-polymer retainers in place of the positively charged microfiltration membrane, and except for the feed stream being an IgG-containing cell culture supernatant with no extra salt added.

### Example 14

The procedure of Example 13 was reproduced except substituting a negatively charged membrane (Sartobind S nano) for the iminodiacetic acid monolith. The results were the same as Example 12.

### Example 15

The procedure of Example 14 was reproduced except mixing porous styrene divinyl benzene particles bearing the negatively charged chelating ligand iminodiacetic acid (Chelex 100) with the positively charged tris(2-aminoethyl)amine chelating ligand agarose particles. The mixed particles were sandwiched between porous polyethylene retainers, and IgG-containing cell culture supernatant treated with 1% allantoin and 0.025% ethacridine was flowed over them. Aggregates were removed. IgG recovery was 99%. It will be apparent to the person of skill that both chemical groups could be mixed on the surface of a single particle type.

### Example 16

The procedure of Example 15 was reproduced except adding a butyl hydrophobic ligand on negatively charged porous polymethacrylate particles (Macroprep T-Butyl, Bio-Rad) to the mixture such that the proportions were 2 parts positively charged particles, to 1 part negative hydrophobic particles, to 1 part negatively charged chelating particles. Antibody recovery was 99% and aggregates were reduced from about 7% to less than 2%. Ethacridine was eliminated.

### Example 17

Monoclonal IgG-containing mammalian cell culture supernatant was treated as described in example 15. Aggregates were reduced from more than 2.7% to 0.31%. IgG recovery was 99%. The antibody was subsequently purified by a 3-step process consisting of steric exclusion chromatography, cation exchange chromatography, and anion exchange chromatography. DNA in the final purified IgG was reduced to such an extent that it could not be measured. Aggregates were reduced to less than 0.05%, essentially the limit of detection for size exclusion chromatography. Final antibody purity was calculated to be greater than 99.999%. Final antibody recovery was 81%.

### Example 18

Filtered supernatant from a suspension of mammalian cells infected with murine leukemia virus was treated with 1% allantoin and 0.025% ethacridine, and then passed over the monoliths as described in example 10. Virus levels were reduced by 3.06 logs. DNA was reduced by 3.5 logs. Ethacridine was eliminated from the treated sample.

### Example 19

The procedure of Example 17 was reproduced, except with filtered supernatant from a suspension of mammalians cells infected with minute virus of mice. Virus levels were reduced by 5.06 logs. DNA was reduced by 3.5 logs. Ethacridine was eliminated from the treated sample. These last two examples illustrate that the invention has valuable utility for reducing virus contamination, as well as for removing aggregates, and host-derived contaminants. It will be apparent to the person of skill that it will also remove endotoxin.

It will be apparent to the person of skill that the above examples employ chemical groups on particles, monoliths, membranes, and fibers; and in combinations of positively charged chelators with negatively charged groups, positively charged chelators with negatively charged chelators, positively charged groups with negatively charged chelators, and both combined with other chemical reactivities such as hydrophobic interaction ligands. Despite the diversity of these examples with IgG and IgM antibodies, it will be further apparent that many more such combinations could be conceived which, though different, manifest the essential features of the invention, and that it may be applied to many types of proteins other than antibodies where it can be expected to produce benefits similar to those illustrated above.

The present invention may be combined with various purification methods to achieve the desired levels of purification. Examples include, but are not limited to, other methods commonly used for purification of antibodies, such as protein A and other forms of affinity chromatography, anion exchange chromatography, cation exchange chromatography, hydrophobic interaction chromatography, immobilized metal affinity chromatography, and additional mixed mode chromatography methods. It is within the purview of a person of ordinary skill in the art to develop appropriate conditions for the various methods and integrate them with the invention herein to achieve the necessary purification of a particular antibody.

All references cited herein are incorporated by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

All numbers expressing quantities of ingredients, chromatography conditions, and so forth used in the specification and claims are to be understood as being modified in all instances by the term “about.” Accordingly, unless indicated to the contrary, the numerical parameters set forth in the specification and attached claims are approximations that may vary depending upon the desired performance sought to be obtained by the present invention.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

1. A composition of matter for reducing the aggregate content of a protein preparation, comprising a first substrate comprising a first surface-bound ligand possessing a metal affinity functionality and a second surface-bound ligand optionally provided on a second substrate and having an aggregate charge opposite to that of the metal affinity functionality of the first substrate, wherein the first surface-bound ligand and the second surface-bound ligand are positioned such that the protein preparation may contact both the first surface-bound ligand and the second surface-bound ligand simultaneously.

2. The composition of claim 1, wherein the first surface-bound ligand, the second surface-bound ligand, or both comprise a multidentate metal chelating moiety.

3. The composition of claim 1, wherein the second surface-bound ligand possesses metal affinity functionality.

4. The composition of claim 1, wherein the first surface-bound ligand has an aggregate charge which is electronegative.

5. The composition of claim 4, wherein the first substrate has additional chemical moieties bound to it, provided that an aggregate charge of the first substrate remains electronegative.

6. The composition of claim 1, wherein the first surface-bound ligand is electronegative and selected from the group consisting of iminodiacetic acid (2-(carboxymethylamino)acetic acid), ethylene glycol(aminoethylether)diacetic acid, nitriloacetic acid (2,2′,2″-nitrilotriacetic acid), aspartic acid, and glutamic acid.

7. The composition of claim 1, wherein the second surface-bound ligand is tris(2-aminoethyl)amine, diethylenetriamine, triethylenetetramine, tetraethylenepentamine, polypropylenimine tetraamine, or desferroxamine.

8. The composition of claim 1, wherein the first surface-bound ligand is iminodiacetic acid (2-(carboxymethylamino)acetic acid).

9. The composition of claim 1, wherein the first surface-bound ligand has an aggregate charge which is electropositive.

10. The composition of claim 1, wherein the first substrate has additional chemical moieties bound to it, provided that an aggregate charge of first substrate remains electropositive.

11. The composition of claim 1, wherein the first surface-bound ligand is tris(2-aminoethyl)amine, diethylenetriamine, triethylenetetramine, tetraethylenepentamine, polypropylenimine tetraamine, or desferroxamine.

12. The composition of claim 1, wherein at least one of the first substrate or optional second substrate has one or more further surface-bound ligands in addition to the first surface-bound ligand and the second surface-bound ligand, wherein the one or more further surface-bound ligands enhance the capacity of the composition to participate in hydrogen bonding, hydrophobic interactions, or pi-pi binding with a component of the protein preparation.

13. The composition of claim 1, wherein the first substrate, the second substrate, or both comprise more than one negatively charged metal chelating group in combination with one or more oppositely charged groups.

14. The composition of claim 1, wherein the first substrate, the second substrate, or both comprise more than one positively charged metal chelating group in combination with one or more oppositely charged groups.

15. The composition of claim 1, wherein the first surface-bound ligand and the second surface-bound ligand are covalently bound to the first substrate.

16. The composition of claim 1, wherein the first surface-bound ligand is covalently bound to the first substrate and the second surface-bound ligand covalently bound to the second substrate which is different from the first substrate.

17. The composition of claim 1 wherein the first substrate, second substrate, or both comprise particles.

18. The composition of claim 17, wherein the particles are non-porous.

19. The composition of claim 17, wherein the particles are porous.

20. The composition of claim 17, wherein the particles have a pore size large enough to permit entry of a protein in a protein preparation.

21. The composition of claim 17, wherein the particles have a pore size too small to permit entry of a protein in a protein preparation.

22. The composition of claim 19, wherein the particles have an average pore size between about 10 nm and about 100 nm.

23. The composition of claim 19, wherein the particles have an average pore size less than about 10 nm.

24. The composition of claim 19, wherein the particles have an average pore size more than about 100 nm.

25. The composition of claim 1, wherein the first substrate, second substrate, or both, comprise a membrane, or a monolith.

26. The composition of any one of claims 1 to 12, wherein the first substrate, second substrate, or both comprise a solid or porous walled hollow fiber.

27. An apparatus comprising the composition of any one of claims 1 to 26, wherein the apparatus is optionally configured for chromatography.

28. The apparatus of claim 27, further configured to allow the protein preparation to subsequently contact, in any order, the composition according to any one of claims 1 to 26 and one or more further substrates comprising one or more further surface-bound ligands comprising a charge selected from the group consisting of electronegative, electropositive, neutral, and combinations thereof.

29. The apparatus of any one of claim 27 or 28, wherein the apparatus is a chromatographic device packed with the first substrate, optional second substrate, one or more further substrates, and combinations thereof.

30. The apparatus of any one of claims 27 to 29, wherein a composition at an entrance of the apparatus is different from a composition at the exit of the apparatus.

31. The apparatus of any one of claims 27 to 30, wherein a chemical surface of one or more components of the apparatus is substantially inert or of sufficiently low surface area as to make no significant contribution to the chemical functionality of the apparatus.

32. The apparatus of any one of claims 27 to 31, wherein the chemical surface of one or more components is configured to allow one or more of (1) create structural integrity, (2) direct flow of liquids therethrough, and (3) physically block, entrap, or entrain insoluble materials in a protein preparation to prevent them from interfering with the effective use of the device.

33. The apparatus of any one of claims 27 to 32, wherein the apparatus comprises one or more porous membranes and at least one of the one or more porous membranes is the first substrate to which the first surface-bound ligand or the second surface-bound ligand is bound.

34. The composition of any one of claims 27 to 32, wherein the first substrate, optional second substrate, or both comprise a permeable chopped fiber mat.

35. The composition of any one of claims 27 to 32, wherein the first substrate, optional second substrate, or both comprise an ordered wound, woven, or non-woven fiber structure.

36. The composition of any one of claims 27 to 32, wherein the first substrate, optional second substrate, or both comprise a combination of a permeable chopped fiber mat and an ordered wound, woven, or non-woven fiber structure.

37. The apparatus of any one of claims 27 to 32, wherein the apparatus comprises porous reticular fibers, wherein the porous reticular fibers are hollow porous-walled fibers or non-porous fibers, and wherein the porous reticular fibers are the first substrate to which the first surface-bound ligand and the second surface-bound ligand are bound.

38. The apparatus of any one of claims 27 to 32, wherein the apparatus comprises porous or non-porous particles sandwiched between porous membranes, or monoliths.

39. The apparatus of any one of claims 27 to 32, wherein porous or non-porous particles are the first substrate to which either the first surface-bound ligand or the second surface-bound ligand is bound and a membrane or monolith is a second substrate to which the other of the first surface-bound ligand and the second surface-bound ligand is bound.

40. The apparatus of any one of claims 27 to 32, wherein the apparatus comprises porous or non-porous particles sandwiched between a woven or amorphous fibrous filter, or embedded within a fibrous matrix.

41. The apparatus of any one of claims 27 to 32, wherein particles comprise the first substrate or second substrate to which either the first surface-bound ligand or the second surface-bound ligand is bound and fibrous filters comprise the second substrate to which the other of the first surface-bound ligand and the second surface-bound ligand is bound.

42. The apparatus of any one of claims 27 to 32, wherein the apparatus comprises porous or non-porous particles sandwiched between woven or crystalline frits.

43. The apparatus of any one of claims 27 to 32, wherein the porous or non-porous particles are the first substrate to which either the first surface-bound ligand or the second surface-bound ligand is bound and the woven or crystalline frits are the second substrate to which the other of the first surface-bound ligand and the second surface-bound ligand is bound.

44. The apparatus of any one of claims 27 to 32 wherein the apparatus comprises porous or non-porous particles embedded in a reticular polymer network.

45. The apparatus of any one of claims 27 to 44, wherein the first substrate and second substrate comprise any combination of configurations as recited in claims 27-44.
